(19) United States
(12) Patent Application Publication
(10) Pub. No.: US 2013/0225870 A1
(43) Pub. Date: Aug. 29, 2013

(54) METHOD OF SYNTHESIZING SUBSTITUTED 2-ALKYL PHENOLS

(71) Applicants: Chi-Wan Lee, Grafton, MA (US); Steven Bloom, Sudbury, MA (US)

(72) Inventors: Chi-Wan Lee, Grafton, MA (US); Steven Bloom, Sudbury, MA (US)

(73) Assignee: SYNTA PHARMACEUTICALS CORP., Lexington, MA (US)

(21) Appl. No.: 13/818,574

(22) PCT Filed: Aug. 25, 2010
(86) PCT No.: PCT/US10/46641

§ 371 Date: May 7, 2013

(51) Int. Cl. C07C 41/18; C07C 37/50

(52) U.S. Cl. CPC C07C 41/18; C07C 37/50
USPC 568/650; 568/772

(57) ABSTRACT

Methods of synthesizing 4-alkyl resorcinols and other substituted phenol compounds, according to formula (IV): or salts thereof, are disclosed, wherein the variables are defined herein.

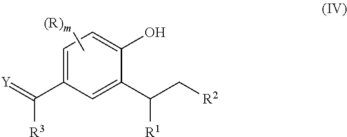

### BACKGROUND OF THE INVENTION

Resorcinol and its derivatives have a wide variety of applications, from the cosmetic industry to pharmaceuticals. Commonly, contract vendors for synthesizing 4-isoalkyl resorcinols use methods such as disclosed in WO 04/52814 (Unilever). However, this method produces five side products and is expensive. In order to isolate the desired 4-isoalkyl resorcinol from the side products produced by these methods, a complicated and expensive purification procedure is required. Typically the purification requires a long and tedious fractional distillation process which limits the number of commercial vendors that can supply 4-isoalkyl resorcinol and increases the cost of making the common starting material.

There is a clear need for a cheaper and more robust synthesis of 4-isoalkyl resorcinol compounds for use in the pharmaceutical and cosmetic industries.

### SUMMARY OF THE INVENTION

Improved methods for synthesizing 4-isopropyl resorcinol (“4-IPR”) compounds, as well as other substituted phenols, starting from a substituted coumarin, are described herein. The resulting process is believed to be cheaper, utilizes milder reaction conditions, uses commodity chemicals, and/or allows for easier purification than previous procedures.

One embodiment provides a method of preparing a compound according to Formula (II) or a salt thereof, wherein a compound of Formula (I):

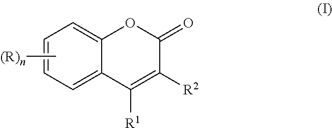

is reacted with a hydroxide-containing base in an organic solvent while heating, thereby producing a compound of Formula (II):

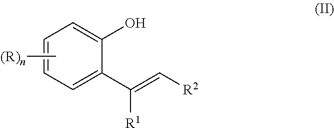

wherein each of R¹ and R² is independently H, C₁-C₆ alkyl, C₃-C₇ cycloalkyl, 5-10 membered heterocyclyl, C₆-C₁₀ aryl or a 5-10 membered heteroaryl; each R is independently —Cl, —Br, —I, C₁-C₆ alkyl, —OR⁵, —C(O)NR⁵, —NR⁵C(O)R⁵, —NO₂, —N(R⁵)₂, or —S(O)ₚR⁵; each R⁵ is independently —H, C₁-C₆ alkyl, C₃-C₇ cycloalkyl, 5-7 membered heterocyclyl, C₆-C₁₀ aryl, 5-7 membered heteroaryl, or a protecting group; p is 0, 1 or 2; and n is 0, 1, 2, or 3; provided that the compound of formula (I) is not 4,7-dimethylcoumarin.

In the compounds according to formulae (I) and (II), each alkyl, cycloalkyl, heterocyclyl, aryl, and heteroraryl represented by R, R¹, R² or R⁵ is independently and optionally substituted with one or more substituents selected from the group consisting of: halo, cyano, nitro, azido, C₁-C₃ alkyl, C₁-C₃ haloalkyl, C₁-C₃ alkoxy, C₁-C₃ haloalkoxyl, C₃-C₆ cycloalkyl, 5-7 membered heterocyclyl, oxo (═O), thiooxo (═S), imino (═N), —C(O)R¹⁰, —C(O)OR¹⁰, —C(O)N(R¹⁰)₂, —N(R¹⁰)₂, and —NR¹⁰C(O)R¹⁰; and each R¹⁰ is independently H or C₁-C₄ alkyl; or two R¹⁰ moieties attached to a single nitrogen atom are taken together to form a 5-7 membered heterocyclyl or heteroaryl.

In another embodiment, a method of preparing 4-isopropyl resorcinol, a compound according to Formula (IIIb) or a salt thereof, including:

a) reacting a compound of Formula (Ib):

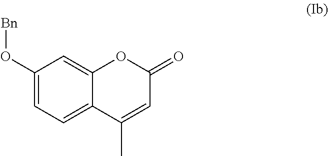 [SMILES: Cc1cc(=O)oc2cc(OCc3ccccc3)ccc12]

with a hydroxide-containing base in an organic solvent, thereby producing a compound of Formula (IIb):

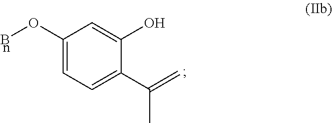 [SMILES: C=C(C)c1ccc(OCc2ccccc2)cc1O]

and

b) hydrogenating the double bond of formula (IIb) and deprotecting the benzyloxy to form a compound of Formula (IIIb):

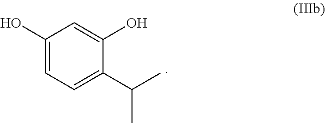

In an embodiment, a method of preparing a compound according to Formula (VIb) or a salt thereof includes:

a) reacting a compound of Formula (Ib):

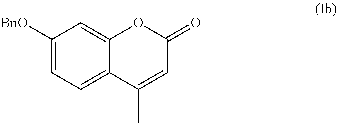 [SMILES: Cc1cc(=O)oc2cc(OCc3ccccc3)ccc12]

with a base in an organic solvent, thereby producing a compound of Formula (IIb):

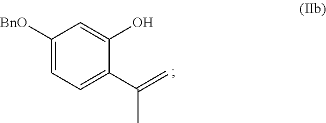 [SMILES: C=C(C)c1ccc(OCc2ccccc2)cc1O ; ";"]

b) hydrogenating the double bond of formula (IIb) and deprotecting the benzyloxy to form a compound of Formula (IIIb):

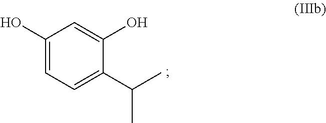

and

c) reacting the compound of Formula (III) with DMF to produce a compound of Formula (IVb):

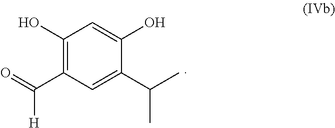

### DETAILED DESCRIPTION OF THE INVENTION

### Definitions

Unless otherwise specified, the below terms used herein are defined as follows:

As used herein, the term “alkyl” means a saturated or unsaturated, straight chain or branched, non-cyclic hydrocarbon having from 1 to 10 carbon atoms. Representative straight chain alkyls include methyl, ethyl, ethenyl, ethynyl, n-propyl, propenyl, propynyl, n-butyl, butenyl, butyryl, n-pentyl, pentenyl, pentynyl, n-hexyl, hexenyl, hexynyl, n-heptyl, heptenyl, heptynyl, n-octyl, octenyl, octynyl, n-nonyl, nonenyl, nonynyl, n-decyl, decenyl, and decynyl; while representative branched alkyls include isopropyl, sec-butyl, isobutyl, tent-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, and the like. The term “(C₁-C₆)alkyl” means a saturated or unsaturated, straight chain or branched, non-cyclic hydrocarbon having from 1 to 6 carbon atoms. Alkyl groups may be optionally substituted with one or more substituents.

As used herein, the term “cycloalkyl” means a saturated or unsaturated, mono- or polycyclic, non-aromatic hydrocarbon having from 3 to 20 carbon atoms. Representative cycloalkyls include cyclopropyl, 1-methylcyclopropyl, cyclopropenyl, cyclobutyl, cyclobutenyl, cyclopentyl, cyclopentenyl, cyclohexyl, cyclohexenyl, cycloheptyl, cyclooctyl, cyclooctenyl, cyclononyl, cyclodecyl, octahydropentalenyl, and the like. Cycloalkyl groups may be optionally substituted with one or more substituents.

As used herein, an “alkoxy” is an alkyl group which is attached to another moiety via an oxygen linker. Alkoxy groups may be optionally substituted with one or more substituents.

As used herein, a “haloalkoxy” is an alkyl group substituted with one or more halogen atoms which is attached to another moiety via an oxygen linker.

As used herein, the term “aryl” means a mono- or polycyclic hydrocarbon, containing from 6 to 10 carbon atoms, in which at least one ring is aromatic. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Aryl groups may be optionally substituted with one or more substituents. In one embodiment, the aryl group is a monocyclic ring, wherein the ring comprises 6 carbon atoms, referred to herein as “(C₆)aryl.”

As used herein, the term “heterocyclyl” means a monocyclic or a polycyclic, saturated or unsaturated, non-aromatic ring or ring system which typically contains 5- to 20-members and at least one heteroatom. A heterocyclic ring system can contain saturated ring(s) or unsaturated non-aromatic ring(s), or a mixture thereof. A 5- to 10-membered heterocycle can contain up to 5 heteroatoms, and a 7- to 20-membered heterocycle can contain up to 7 heteroatoms. Typically, a heterocycle has at least one carbon atom ring member. Each heteroatom is independently selected from nitrogen, which can be oxidized (e.g., N(O)) or quaternized, oxygen and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, a nitrogen atom may be substituted with a tert-butoxycarbonyl group. Furthermore, the heterocyclyl group may be optionally substituted with one or more substituents.

As used herein, the term “heteroaromatic”, “heteroaryl”, or like terms, means a monocyclic or a polycyclic, unsaturated radical containing at least one heteroatom, in which at least one ring is aromatic. Polycyclic heteroaryl rings must contain at least one heteroatom, but not all rings of a polycyclic heteroaryl moiety must contain heteroatoms. Each heteroatom is independently selected from nitrogen, which can be oxidized (e.g., N(O)) or quaternized, oxygen and sulfur, including sulfoxide and sulfone. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, an isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzothienyl. In one embodiment, the heteroaromatic ring is selected from 5-8 membered monocyclic heteroaryl rings. The point of attachment of a heteroaromatic or heteroaryl ring may be at either a carbon atom or a heteroatom. Heteroaryl groups may be optionally substituted with one or more substituents. As used herein, the term “(C₅)heteroaryl” means an heteroaromatic ring of 5 members, wherein at least one carbon atom of the ring is replaced with a heteroatom, such as, for example, oxygen, sulfur or nitrogen. Representative (C₅)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like. As used herein, the term “(C₆)heteroaryl” means an aromatic heterocyclic ring of 6 members, wherein at least one carbon atom of the ring is replaced with a heteroatom such as, for example, oxygen, nitrogen or sulfur. Representative (C₆)heteroaryls include pyridyl, pyridazinyl, pyrazinyl, triazinyl, tetrazinyl, and the like. Furthermore, a heteroaryl group may be optionally substituted with one or more substituents.

As used herein, the term “halogen” or “halo” means —F, —Cl, —Br or —I.

The term “hydroxide-containing base” encompasses any compound containing a hydroxide (—OH) moiety with a pH above 7. Examples of hydroxide-containing bases include, without limitation, NaOH, KOH, CsOH, etc.

The term “organic solvent”encompasses any polar or non-polar, protic or aprotic liquid. Examples of common organic solvents include, without limitation, DMF, methylene chloride, ethyl acetate, ethanol, THF, etc.

Unless indicated otherwise, compounds containing reactive functional groups, such as, for example, carboxy, hydroxy, thiol and amino moieties, also include corresponding protected derivatives thereof “Protected derivatives” are those compounds in which a reactive site or sites are blocked with one ore more protecting groups. Compounds according to Formulae (I)-(V) may include “protecting groups.” Examples of suitable protecting groups for hydroxyl groups include benzyl, methoxymethyl, allyl, trimethylsilyl, tert-butyldimethylsilyl, acetate, and the like. Examples of suitable amine protecting groups include benzyloxycarbonyl, tert-butoxycarbonyl, tert-butyl, benzyl and fluorenylmethyloxy-carbonyl (Fmoc). Examples of suitable thiol protecting groups include benzyl, tert-butyl, acetyl, methoxymethyl and the like. Other suitable protecting groups are well known to those of ordinary skill in the art and include those found in T. W. GREENE, PROTECTING GROUPS IN ORGANIC SYNTHESIS, (John Wiley & Sons, Inc., 1981).

The compounds described herein include those of Formulae (I)-(V), or salts, clathrates, solvates, or hydrates thereof; and protected derivatives of compounds of Formulae (I)-(V).

The compounds described herein may contain one or more chiral centers and/or double bonds and, therefore, exist as stereoisomers, such as double-bond isomers (i.e., geometric isomers), enantiomers or diastereomers. According to this invention, the chemical structures depicted herein, including the compounds of this invention, encompass all of the corresponding compounds' enantiomers, diastereomers and geometric isomers, that is, both the stereochemically pure form (e.g., geometrically pure, enantiomerically pure, or diastereomerically pure) and isomeric mixtures (e.g., enantiomeric, diastereomeric and geometric isomeric mixtures). In some cases, one enantiomer, diastereomer or geometric isomer will possess superior activity or an improved toxicity or kinetic profile compared to other isomers. In those cases, such enantiomers, diastereomers and geometric isomers of compounds of this invention are preferred.

As used herein, the term “pharmaceutically acceptable salt” or “salt” refers to a salt prepared from a compound of Formulae (I)-(V), or Table 1, having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. “Pharmaceutically acceptable salt” also refers to a salt prepared from a compound of Formulae (I)-(V) having a basic functional group, such as an amine functional group, and a pharmaceutically acceptable inorganic or organic acid. Suitable acids include, but are not limited to, hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, isonicotinic acid, oleic acid, tannic acid, pantothenic acid, saccharic acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, pamoic acid and p-toluenesulfonic acid.

As used herein, a composition that “substantially” comprises a compound means that the composition contains more than about 80% by weight, more preferably more than about 90% by weight, even more preferably more than about 95% by weight, and most preferably more than about 97% by weight of the compound.

As used herein, a reaction that is “substantially complete” means that the reaction contains more than about 80% by weight of the desired product, more preferably more than about 90% by weight of the desired product, even more preferably more than about 95% by weight of the desired product, and most preferably more than about 97% by weight of the desired product.

As used herein, a “racemic mixture” means about 50% of one enantiomer and about 50% of is corresponding enantiomer relative to a chiral center in the molecule. The invention encompasses all enantiomerically pure, enantiomerically enriched, diastereomerically pure, diastereomerically enriched, and racemic mixtures of the compounds of the invention.

Enantiomeric and diastereomeric mixtures can be resolved into their component enantiomers or diastereomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically pure intermediates, reagents, and catalysts by well known asymmetric synthetic methods.

The compounds described herein are defined herein by their chemical structures and/or chemical names. Where a compound is referred to by both a chemical structure and a chemical name, and the chemical structure and chemical name conflict, the chemical structure is determinative of the compound's identity.

As used herein, a composition that is “substantially free” of a compound means that the composition contains less than about 20% by weight, more preferably less than about 10% by weight, even more preferably less than about 5% by weight, and most preferably less than about 3% by weight of the compound.

In a first embodiment, a method of preparing a compound according to Formula (II), or a salt thereof, comprises the steps of:

reacting a compound of Formula (I):

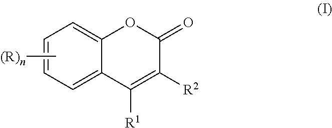

with a hydroxide-containing base in an organic solvent while heating, and thereby producing a compound of Formula (II):

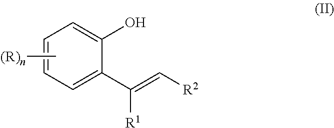

wherein each of R¹ and R² is independently H, C₁-C₆ alkyl, C₃-C₇ cycloalkyl, 5-10 membered heterocyclyl, C₆-C₁₀ aryl or a 5-10 membered heteroaryl; each R is independently —Cl, —Br, —I, C₁-C₆ alkyl, —OR⁵, —C(O)NR⁵, —NR⁵C(O)R⁵, —NO₂, —N(R⁵)₂, or —S(O)ₚR⁵; each R⁵ is independently —H, C₁-C₆ alkyl, C₃-C₇ cycloalkyl, 5-7 membered heterocyclyl, C₆-C₁₀ aryl, 5-7 membered heteroaryl, or a protecting group; p is 0, 1 or 2; and n is 0, 1, 2, or 3; wherein each alkyl, cycloalkyl, heterocyclyl, aryl, and heteroraryl represented by R, R¹, R² or R⁵ is independently and optionally substituted with one or more substituents selected from the group consisting of: halo, cyano, nitro, azido, C₁-C₃ alkyl, C₁-C₃ haloalkyl, C₁-C₃ alkoxy, C₁-C₃ haloalkoxyl, C₃-C₆ cycloalkyl, 5-7 membered heterocyclyl, oxo (═O), thiooxo (═S), imino (═N), —C(O)R¹⁰, —C(O)OR¹⁰, —C(O)N(R¹⁰)₂, —N(R¹⁰)₂, and —NR¹⁰C(O)R¹⁰; and each R¹⁰ is independently H or C₁-C₄ alkyl; or two R¹⁰ moieties attached to a single nitrogen atom are taken together to form a 5-7 membered heterocyclyl or heteroaryl. In one aspect of this embodiment, the compound of formula (I) is not 4,7-dimethylcoumarin.

A second embodiment relates to a method of preparing a compound according to Formula (IVa), or a salt thereof, comprising the steps of:

a) reacting a compound of Formula (I):

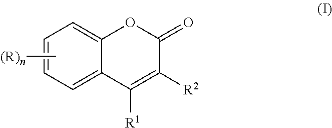

with a base in an organic solvent, and thereby producing a compound of Formula (II):

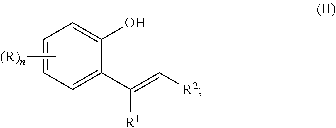

and

b) reacting the compound of Formula (II) with a carbonyl or thiocarbonyl source to produce a compound of Formula (IVa):

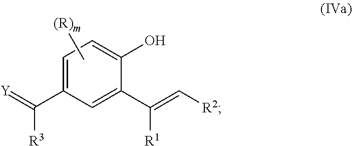

wherein Y is O or S; each of R¹ and R² is independently H, C₁-C₆ alkyl, C₃-C₇ cycloalkyl, 5-10 membered heterocyclyl, C₆-C₁₀ aryl or a 5-10 membered heteroaryl; each R is independently —Cl, —Br, —I, C₁-C₆ alkyl, —OR⁵, —C(O)NR⁵, —NR⁵C(O)R⁵, —NO₂, —N(R⁵)₂, or —S(O)ₚR⁵; R³ is H, OH or SH; each R⁵ is independently —H, C₁-C₆ alkyl, C₃-C₇ cycloalkyl, 5-7 membered heterocyclyl, C₆-C₁₀ aryl, 5-7 membered heteroaryl, or a protecting group; p is 0, 1 or 2; n is 0, 1, 2, or 3; and m is 0, 1 or 2; wherein each alkyl, cycloalkyl, heterocyclyl, aryl, and heteroraryl represented by R, R¹, R² or R⁵ is independently and optionally substituted with one or more substituents selected from the group consisting of: halo, cyano, nitro, azido, C₁-C₃ alkyl, C₁-C₃ haloalkyl, C₁-C₃ alkoxy, C₁-C₃ haloalkoxyl, C₃-C₆ cycloalkyl, 5-7 membered heterocyclyl, oxo (═O), thiooxo (═S), imino (═N), —C(O)R¹⁰, —C(O)OR¹⁰, —C(O)N(R¹⁰)₂, —N(R¹⁰)₂, and —NR¹⁰C(O)R¹⁰; and each R¹⁰ is independently H or C₁-C₄ alkyl; or two R¹⁰ moieties attached to a single nitrogen atom are taken together to form a 5-7 membered heterocyclyl or heteroaryl.

In one aspect of the above first or second methods the reaction of a compound of formula (I) occurs in an organic solvent has a boiling point of about 100° C. or above. In a particular embodiment, the organic solvent is selected from xylene, toluene, an alcohol, DMA, DME, NMP, DMF, ethylene glycol, diethylene glycol.

In one aspect of the above first or second methods, the hydroxide-containing base in the reaction of a compound of formula (I) is NaOH or KOH.

In one aspect of the above methods, the reaction of a compound of formula (I) is heated to between about 100° C. to about 125° C.

In one aspect of the above first or second methods, the reaction of a compound of formula (I) is done using the following conditions: the hydroxide-containing base is NaOH, the organic solvent is DMF, and the reaction temperature is about 110° C.

In one aspect of the second method, the reaction in step b) is a Vilsmeire-Haack reaction; and the compound of Formula (IVa) is wherein Y is O and R³ is H. In a more particular embodiment of this method, the reaction in step b) is run at a temperature range of about −10° C. to about 5° C. In a more particular embodiment of this method, the reaction in step b) is performed using POCl₃ in an organic solvent. In a more particular embodiment of this method, the organic solvent is DMF. In a more particular embodiment of this method, the reaction is performed in an inert atmosphere. In a more particular embodiment, the inert atmosphere is nitrogen gas or argon gas.

In one aspect of the second method, the reaction in step b) produces a compound according to Formula (IV) wherein Y is S and R³ is SH. In one embodiment of this method, the compound of Formula (II) in step b) is reacted with potassium ethyl xanthate in an organic solvent with heating. In a more particular embodiment of this method, the organic solvent in step b) is DMF. In a more particular embodiment of this method, the reaction in step b) is performed at about 100° C.

A third embodiment includes a method of preparing a compound according to Formula (III), or a salt thereof, comprising the steps of:

a) reacting a compound of Formula (I):

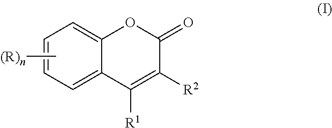

with a hydroxyide containing base in an organic solvent while heating, and thereby producing a compound of Formula (II):

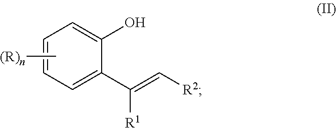

and

b) reducing the double bond of the compound of Formula (II), thereby producing a compound of Formula (III):

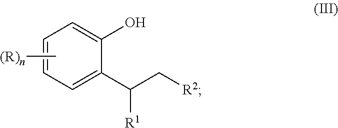

wherein, each of R¹ and R² is independently H, C₁-C₆ alkyl, C₃-C₇ cycloalkyl, 5-10 membered heterocyclyl, C₆-C₁₀ aryl or a 5-10 membered heteroaryl; each R is independently —Cl, —Br, —I, C₁-C₆ alkyl, —OR⁵, —C(O)NR⁵, —NR⁵C(O)R⁵, —NO₂, —N(R⁵)₂, or —S(O)ₚR⁵; each R⁵ is independently —H, C₁-C₆ alkyl, C₃-C₇ cycloalkyl, 5-7 membered heterocyclyl, C₆-C₁₀ aryl, 5-7 membered heteroaryl, or a protecting group; p is 0, 1 or 2; and n is 0, 1, 2, or 3; wherein each alkyl, cycloalkyl, heterocyclyl, aryl, and heteroraryl represented by R, R¹, R² or R⁵ is independently and optionally substituted with one or more substituents selected from the group consisting of: halo, cyano, nitro, azido, C₁-C₃ alkyl, C₁-C₃ haloalkyl, C₁-C₃ alkoxy, C₁-C₃ haloalkoxyl, C₃-C₆ cycloalkyl, 5-7 membered heterocyclyl, oxo (═O), thiooxo (═S), imino (═N), —C(O)R¹⁰, —C(O)OR¹⁰, —C(O)N(R¹⁰)₂, —N(R¹⁰)₂, and —NR¹⁰C(O)R¹⁰; and each R¹⁰ is independently H or C₁-C₄ alkyl; or two R¹⁰ moieties attached to a single nitrogen atom are taken together to form a 5-7 membered heterocyclyl or heteroaryl.

A fourth embodiment includes a method of preparing a compound according to Formula (IV), or a salt thereof, comprising the steps of:

a) reacting a compound of Formula (I):

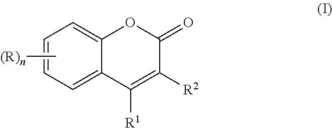

with a base in an organic solvent, and thereby producing a compound of Formula (II):

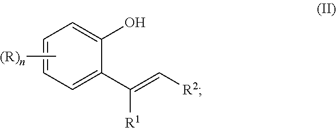

and

b) reducing the double bond of the compound of Formula (II), thereby producing a compound of Formula (III):

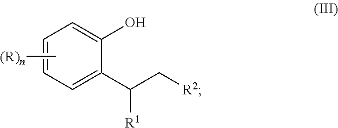

and

c) reacting the compound of Formula (III) with a carbonyl or thiocarbonyl source to produce a compound of Formula (IV):

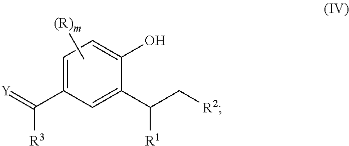

wherein Y is O or S; each of R¹ and R² is independently H, C₁-C₆ alkyl, C₃-C₇ cycloalkyl, 5-10 membered heterocyclyl, C₆-C₁₀ aryl or a 5-10 membered heteroaryl; each R is independently —Cl, —Br, —I, C₁-C₆ alkyl, —OR⁵, —C(O)NR⁵, —NR⁵C(O)R⁵, —NO₂, —N(R⁵)₂, or —S(O)ₚR⁵; R³ is H, OH or SH; each R⁵ is independently —H, C₁-C₆ alkyl, C₃-C₇ cycloalkyl, 5-7 membered heterocyclyl, C₆-C₁₀ aryl, 5-7 membered heteroaryl, or a protecting group; p is 0, 1 or 2; n is 0, 1, 2, or 3; and m is 0, 1 or 2; wherein each alkyl, cycloalkyl, heterocyclyl, aryl, and heteroraryl represented by R, R¹, R² or R⁵ is independently and optionally substituted with one or more substituents selected from the group consisting of: halo, cyano, nitro, azido, C₁-C₃ alkyl, C₁-C₃ haloalkyl, C₁-C₃ alkoxy, C₁-C₃ haloalkoxyl, C₃-C₆ cycloalkyl, 5-7 membered heterocyclyl, oxo (═O), thiooxo (═S), imino (═N), —C(O)R¹⁰, —C(O)OR¹⁰, —C(O)N(R¹⁰)₂, —N(R¹⁰)₂, and —NR¹⁰C(O)R¹⁰; and each R¹⁰ is independently H or C₁-C₄ alkyl; or two R¹⁰ moieties attached to a single nitrogen atom are taken together to form a 5-7 membered heterocyclyl or heteroaryl.

In a fifth embodiment a method of preparing a compound according to Formula (V), or a salt thereof, comprises the steps of:

a) reacting a compound of Formula (I):

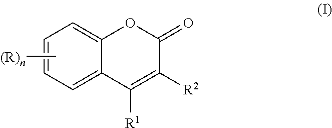

with a base in an organic solvent, and thereby producing a compound of Formula (II):

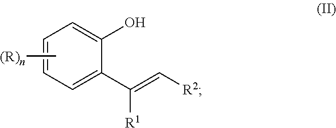

and

b) hydrogenating the double bond of the compound of Formula (II), thereby producing a compound of Formula (III):

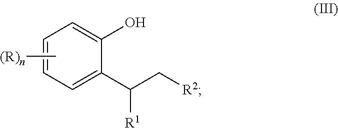

c) reacting the compound of Formula (III) with a carbonyl or thiocarbonyl source to produce a compound of Formula (IV):

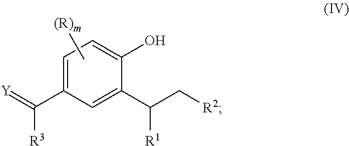

and

d) protecting the free hydroxyl group on the phenyl ring of Formula (IV) to produce a compound of Formula (V):

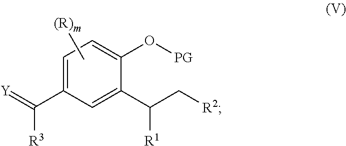

wherein Y is O or S; each of R¹ and R² is independently H, C₁-C₆ alkyl, C₃-C₇ cycloalkyl, 5-10 membered heterocyclyl, C₆-C₁₀ aryl or a 5-10 membered heteroaryl; each R is independently —Cl, —Br, —I, C₁-C₆ alkyl, —OR⁵, —C(O)NR⁵, —NR⁵C(O)R⁵, —NO₂, —N(R⁵)₂, or —S(O)ₚR⁵; R³ is H, OH or SH; each R⁵ is independently —H, C₁-C₆ alkyl, C₃-C₇ cycloalkyl, 5-7 membered heterocyclyl, C₆-C₁₀ aryl, 5-7 membered heteroaryl, or a protecting group; p is 0, 1 or 2; n is 0, 1, 2, or 3; m is 0, 1 or 2; and PG is a protecting group; wherein each alkyl, cycloalkyl, heterocyclyl, aryl, and heteroraryl represented by R, R¹, R² or R⁵ is independently and optionally substituted with one or more substituents selected from the group consisting of: halo, cyano, nitro, azido, C₁-C₃ alkyl, C₁-C₃ haloalkyl, C₁-C₃ alkoxy, C₁-C₃ haloalkoxyl, C₃-C₆ cycloalkyl, 5-7 membered heterocyclyl, oxo (═O), thiooxo (═S), imino (═N), —C(O)R¹⁰, —C(O)OR¹⁰, —C(O)N(R¹⁰)₂, —N(R¹⁰)₂, and —NR¹⁰C(O)R¹⁰; and each R¹⁰ is independently H or C₁-C₄ alkyl; or two R¹⁰ moieties attached to a single nitrogen atom are taken together to form a 5-7 membered heterocyclyl or heteroaryl.

A sixth embodiment provides a method of preparing 4-isopropyl resorcinol, a compound according to Formula (IIIb) or a salt thereof, comprising the steps of:

a) reacting a compound of Formula (Ib):

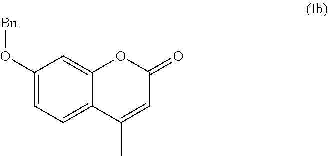 [SMILES: Cc1cc(=O)oc2cc(OCc3ccccc3)ccc12]

with a hydroxide containing base in an organic solvent, and thereby producing a compound of Formula (IIb):

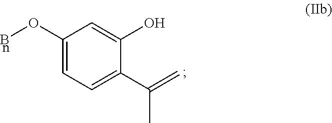 [SMILES: C=C(C)c1ccc(OCc2ccccc2)cc1O ; ";"]

and

b) hydrogenating the double bond of formula (IIb) and deprotecting the benzyloxy to form a compound of Formula (IIIb):

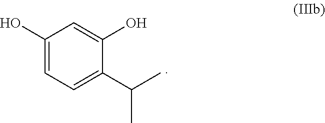

A seventh embodiment provides a method of preparing 2,4-dihydroxy-5-isopropyl benzaldehyde, or a salt thereof, comprising the steps of:

a) reacting a compound of Formula (Ib):

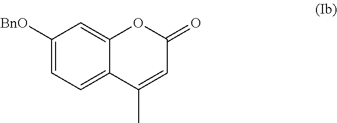 [SMILES: Cc1cc(=O)oc2cc(OCc3ccccc3)ccc12]

with a base in an organic solvent, and thereby producing a compound of Formula (IIb):

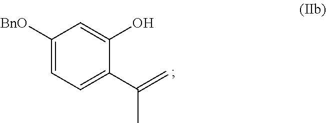 [SMILES: C=C(C)c1ccc(OCc2ccccc2)cc1O ; ";"]

b) hydrogenating the double bond of formula (IIb) and deprotecting the benzyloxy to form a compound of Formula (IIIb):

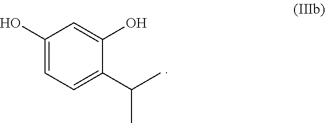

and

c) reacting the compound of Formula (III) with DMF to produce a compound of Formula (IVb):

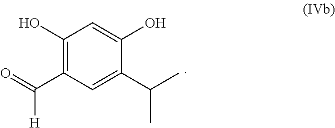

In one aspect of any one of the third through seventh methods, the organic solvent in step a) has a boiling point of about 100° C. or above. More particularly, the organic solvent is selected from xylene, toluene, an alcohol, DMA, DME, NMP, DMF, ethylene glycol, diethylene glycol.

In an aspect of any one of the third through seventh methods, the hydroxide-containing base used in step a) is NaOH or KOH.

In an aspect of any one of the third through seventh methods, the reaction in step a) is heated to between about 100° C. to about 125° C.

In an aspect of any one of the third through seventh methods, in step a) the hydroxide-containing base is NaOH, the organic solvent is DMF, and the reaction temperature is about 110° C.

In an aspect of any one of the third through seventh methods, in step b) the double bond is reduced by any hydrogenation procedure. More particularly, the hydrogenation is done using Pd/C and a source of hydrogen. More particularly, the source of hydrogen is hydrogen gas. More particularly, the source of hydrogen is NH₃CO₂H.

In an aspect of any one of the fourth through seventh methods, the reaction in step c) is a Vilsmeire-Haack reaction; and the compound of Formula (IV) is wherein Y is O and R³ is H. In a more particular aspect, the reaction in step c) is run at a temperature range of about −10° C. to about 5° C. More particularly, the reaction in step c) is performed using POCl₃ in an organic solvent. More particularly, the organic solvent is DMF. More particularly, the reaction is performed in an inert atmosphere. More particularly, the inert atmosphere is nitrogen gas or argon gas.

In an aspect of the fourth or fifth method, the reaction in step c) produces a compound according to Formula (IV) wherein Y is S and R³ is SH. More particularly, the compound of Formula (III) in step c) is reacted with potassium ethyl xanthate in an organic solvent with heating. More particularly, the organic solvent in step c) is DMF. More particularly, the reaction in step c) is performed at about 100° C.

In one aspect of the fifth embodiment, PG is benzyl. In anther aspect of the fifth embodiment, the reaction in step d) is done under mildly basic conditions in an organic solvent. More particularly, the reaction in step d) is performed in DMF using a carbonate base. More particularly, the carbonate base is K₂CO₃.

In one aspect of any of the first through fifth embodiments, the compound according to formula (I) is:

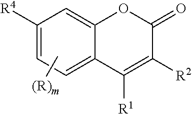

or a salt thereof; wherein R⁴ is —Cl, —Br, —I, C₁-C₆ alkyl, —OR⁵, —C(O)NR⁵, —NR⁵C(O)R⁵, —NO₂, —N(R⁵)₂, or —S(O)ₚR⁵; and m is 0, 1 or 2.

In one aspect of any of the first through fifth embodiments, the compound according to formula (II) is:

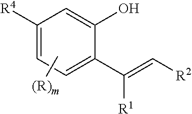

or a salt thereof; wherein R⁴ is —Cl, —Br, —I, C₁-C₆ alkyl, —OR⁵, —C(O)NR⁵, —NR⁵C(O)R⁵, —NO₂, —N(R⁵)₂, or —S(O)ₚR⁵; and m is 0, 1 or 2.

In one aspect of any of the third through fifth embodiments, the compound according to formula (III) is:

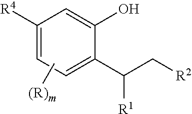

or a salt thereof; wherein R⁴ is —Cl, —Br, —I, C₁-C₆ alkyl, —OR⁵, —C(O)NR⁵, —NR⁵C(O)R⁵, —NO₂, —N(R⁵)₂, or —S(O)ₚR⁵; and m is 0, 1 or 2.

In one aspect of the second embodiment, the compound according to formula (IVa) is:

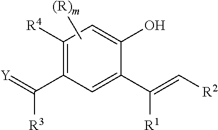

or a salt thereof; wherein R⁴ is —Cl, —Br, —I, C₁-C₆ alkyl, —OR⁵, —C(O)NR⁵, —NR⁵C(O)R⁵, —NO₂, —N(R⁵)₂, or —S(O)ₚR⁵; and m is 0, 1 or 2.

In one aspect of the fourth or fifth embodiments, the compound according to formula (IV) is:

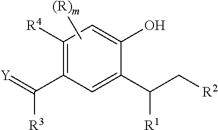

or a salt thereof; wherein R⁴ is —Cl, —Br, —I, C₁-C₆ alkyl, —OR⁵, —C(O)NR⁵, —NR⁵C(O)R⁵, —NO₂, —N(R⁵)₂, or —S(O)ₚR⁵; and m is 0, 1 or 2.

In one aspect of the fifth embodiment, the compound according to formula (V) is:

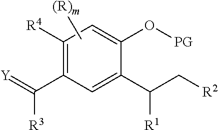

or a salt thereof; wherein R⁴ is —Cl, —Br, —I, C₁-C₆ alkyl, —OR⁵, —C(O)NR⁵, —NR⁵C(O)R⁵, —NO₂, —N(R⁵)₂, or —S(O)ₚR⁵; and m is 0, 1 or 2.

In any of the above particular aspects for the third through fifth embodiments, m is 0 and R⁴ is —OH.

In an aspect of the fifth embodiment, m is 0 and R⁴ is —OPG.

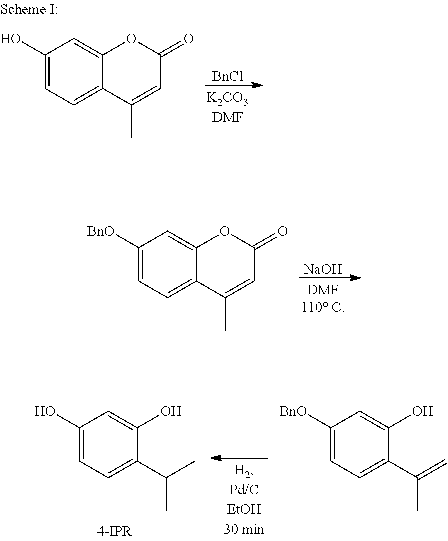

Scheme I details the synthesis of 4-isopropyl resorcinol (4-IPR) starting from 7-hydroxy-4-methyl coumarin.

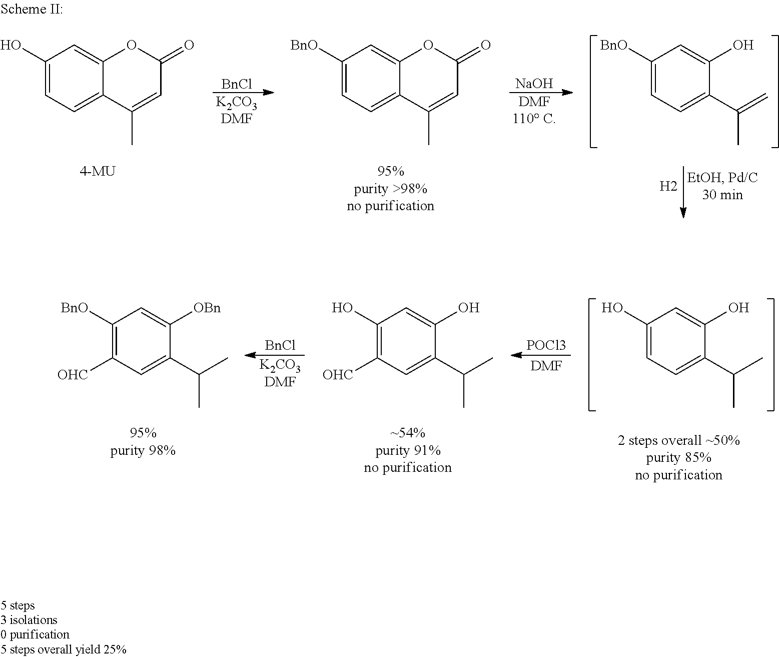

Scheme II shows the overall synthesis of the benzyl protected form of 2,4-dihydroxy-5-isopropyl benzaldehyde starting with 7-hydroxy-4-methyl coumarin. As shown in the scheme, there are no purification steps required between each step, and in fact not all products even need to be isolated. The overall yield for the 5 steps is 25%, and the ending product is 98% pure.

The invention is further defined by reference to the following examples describing in detail the preparation of compounds described herein. It will be apparent to those skilled in the art that many modifications, both to materials and methods, may be practiced without departing from the purpose and interest of the invention described and claimed herein. The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXPERIMENTAL SECTION

### EXAMPLE 1

### Synthesis of 4-IPR

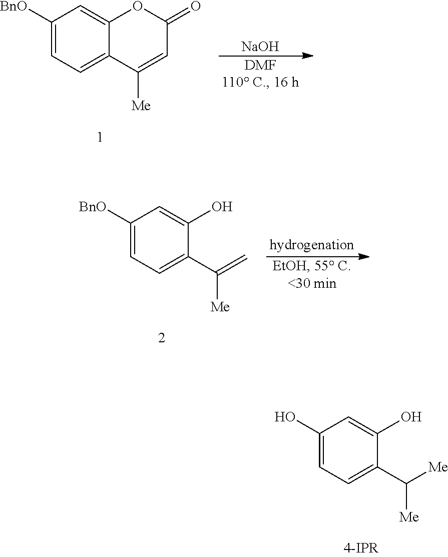

Compound 1 was readily prepared from the reaction of benzyl chloride in the presence of potassium carbonate. Compound 2 was synthesized by hydrolysis/decarboxylation of compound 1 in the presence of sodium hydroxide at 110° C. overnight. Compound 4-IPR was prepared by the catalytic hydrogen transfer reaction with ammonium formate and palladium on carbon in ethyl alcohol in less than 30 min.

For optimization, DMF, ethylene glycol, isopropyl alcohol, isoamyl alcohol, and toluene have been used. NaOH, KOH, CsOH, K₂CO₃, tBuOK have been used as base. Equivalency of base and temperature effect also had been investigated.

### EXAMPLE 2

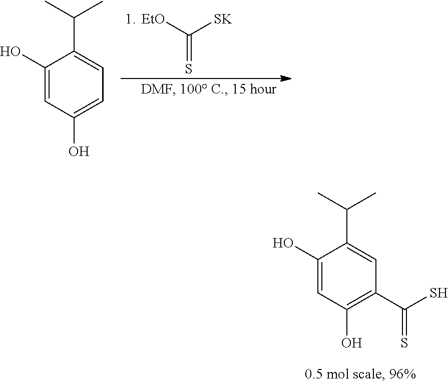

Step 1:

Isopropyl resorcinol (76 g, 0.5 mol, 1.0 equiv.) and potassium ethyl xanthate (100 g, 0625 mol, 1.25 equiv.) were dissolved in 200 mL DMF to from a thick solution. The flask was placed in 100° C. oil bath and stirred for 15 hours under N₂ protection. LC-MS indicated that starting material was consumed. The dark brown solution was poured into 1200 mL ice water with N₂ protection. 6M HCl was added to adjust pH to 2 to 3. An orange solid was formed when solution turned acidic. The solid was collected by filtration, washed with 3×500 mL water, and dried. 110 g of orange-colored solid was isolated, yield 96%.

1.-53. (canceled)

54. A method of preparing a compound according to Formula (II), comprising the steps of:
reacting a compound of Formula (I): 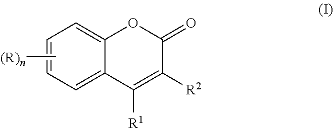
with a hydroxide-containing base in an organic solvent while heating, and thereby producing a compound of Formula (II): 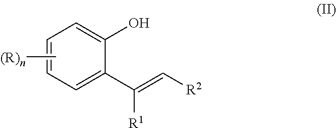
or a salt thereof; wherein each of R¹ and R² is independently H, C₁-C₆ alkyl, C₃-C₇ cycloalkyl, 5-10 membered heterocyclyl, C₆-C₁₀ aryl or a 5-10 membered heteroaryl;
each R is independently —Cl, —Br, —I, C₁-C₆ alkyl, —OR⁵, —C(O)NR⁵, —NR⁵C(O)R⁵, —NO₂, —N(R⁵)₂, or —S(O)ₚR⁵;
each R⁵ is independently —H, C₁-C₆ alkyl, C₃-C₇ cycloalkyl, 5-7 membered heterocyclyl, C₆-C₁₀ aryl, 5-7 membered heteroaryl, or a protecting group; and
p is 0, 1 or 2;
n is 0, 1, 2, or 3;
wherein each alkyl, cycloalkyl, heterocyclyl, aryl, and heteroraryl represented by R, R¹, R² or R⁵ is independently and optionally substituted with one or more substituents selected from the group consisting of: halo, cyano, nitro, azido, C₁-C₃ alkyl, C₁-C₃ haloalkyl, C₁-C₃ alkoxy, C₁-C₃ haloalkoxyl, C₃-C₆ cycloalkyl, 5-7 membered heterocyclyl, oxo (═O), thiooxo (═S), imino (═N), —C(O)R¹⁰, —C(O)OR¹⁰, —C(O)N(R¹⁰)₂, —N(R¹⁰)₂, and —NR¹⁰C(O)R¹⁰; and
each R¹⁰ is independently H or C₁-C₄ alkyl; or two R¹⁰ moieties attached to a single nitrogen atom are taken together to form a 5-7 membered heterocyclyl or heteroaryl;
provided that the compound of formula (I) is not 4,7-dimethylcoumarin.

55. The method of claim 54, wherein the compound of Formula (II) is further reacted with a carbonyl or thiocarbonyl source to produce a compound of Formula (IVa): 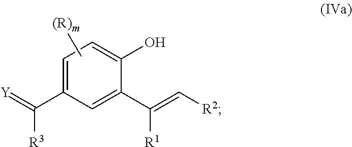
or a salt thereof, wherein
Y is O or S;
R³ is H, OH or SH; and
m is 0, 1 or 2.

56. The Method of claim 54, wherein the double bond of the compound of Formula (II) is reduced to produce a compound of Formula (III): 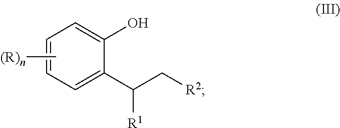
or a salt thereof.

57. The method of claim 56, wherein the compound of Formula (III) is further reacted with a carbonyl source to produce a compound of Formula (IV): 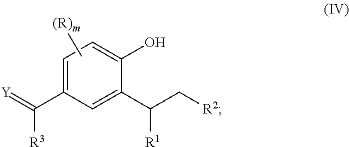
or a salt thereof, wherein
Y is O or S;
R³ is H, OH or SH; and
m is 0, 1 or 2.

58. The method of claim 57, wherein the compound of Formula (IV) is further reacted with a protecting agent to produce a compound of Formula (V): 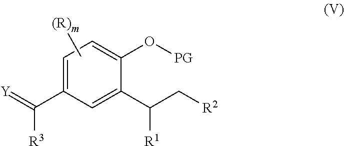
or a salt thereof, wherein PG is a protecting group.

59. A method of preparing a compound according to Formula (IIIb), comprising the steps of:
a) reacting a compound of Formula (Ib): 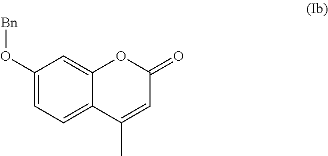 [SMILES: Cc1cc(=O)oc2cc(OCc3ccccc3)ccc12]
with a hydroxide-containing base in an organic solvent, and thereby producing a compound of Formula (IIb): 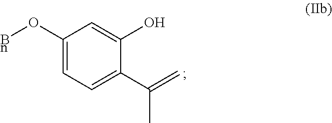 [SMILES: C=C(C)c1ccc(OCc2ccccc2)cc1O]
and
b) hydrogenating the double bond of formula (IIb) and deprotecting the benzyloxy to form a compound of Formula (IIIb): 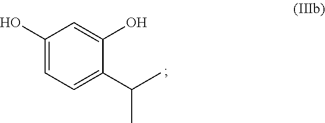
or a salt thereof.

60. The method of claim 59, wherein the compound of Formula (IIIb) is reacted with DMF to produce a compound of Formula (IVb): 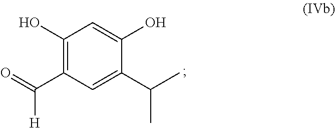
or a salt thereof.

61. The method of claims 54 or 59, wherein the organic solvent has a boiling point of about 100° C. or above.

62. The method of claim 61, wherein the organic solvent is selected from xylene, toluene, an alcohol, DMA, DME, NMP, DMF, ethylene glycol, and diethylene glycol.

63. The method of claims 54 or 59, wherein the base is NaOH or KOH.

64. The method of claims 54 or 59, wherein the base is NaOH, the organic solvent is DMF, and the reaction temperature is about 110° C.

65. The method of claims 56 or 59, wherein the double bond is reduced by a hydrogenation procedure.

66. The method of claim 65, wherein the hydrogenation is done using Pd/C and a source of hydrogen.

67. The method of claim 66, wherein the source of hydrogen is hydrogen gas.

68. The method of claim 66, wherein the source of hydrogen is NH₃CO₂H.

69. The method of claims 55 or 57, wherein the reaction is a Vilsmeire-Haack reaction; and wherein Y is O; and R³ is H for the compound of Formula (IV).

70. The method of claim 60, wherein the reaction is a Vilsmeire-Haack reaction.

71. The method of claims 69 or 70, wherein the reaction is run at a temperature range of about −10° C. to about 5° C.

72. The method of claims 69 or 70, wherein the reaction is performed using POCl₃ in DMF in an inert atmosphere.

73. The method of claim 1, wherein the compound according to formula (I) is: 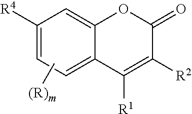
or a salt thereof;
wherein R⁴ is —Cl, —Br, —I, C₁-C₆ alkyl, —OR⁵, —C(O)NR⁵, —NR⁵C(O)R⁵, —NO₂, —N(R⁵)₂, or —S(O)ₚR⁵; and
m is 0, 1 or 2.
